(19) United States
(12) Patent Application Publication
(10) Pub. No.: US 2024/0239753 A1
(43) Pub. Date: Jul. 18, 2024

(54) METHOD FOR CRYSTALLIZATION OF AROMATIC AMINO ACIDS, ENABLING SUSTAINABLE CYCLE OF AMMONIA

(71) Applicant: CJ CheilJedang Corporation, Seoul (KR)

(72) Inventors: Hwayeon LIM, Seoul (KR); Jong Hwan SHIN, Seoul (KR); Hyeongpyo HONG, Seoul (KR); Youn Jae LEE, Seoul (KR); In Sung LEE, Seoul (KR)

(73) Assignee: CJ CheilJedang Corporation, Seoul (KR)

(21) Appl. No.: 18/562,666

(22) PCT Filed: May 20, 2022
(86) PCT No.: PCT/KR2022/007224

§ 371 Date: Nov. 20, 2023

(30) Foreign Application Priority Data
May 21, 2021 (KR) 10-2021-0065600

(51) Int. Cl. C07D 233/64; C07B 63/04; C07C 229/34; C07D 209/20

(52) U.S. Cl. CPC C07D 233/64; C07B 63/04; C07C 229/34; C07D 209/20

(57) ABSTRACT

The present application provides: a method for crystallization of an aromatic amino acid, enabling sustainable cycle of ammonia; and aromatic amino acid crystals produced by the method.

### CROSS-REFERENCE TO RELATED APPLICATION(S)

The present application claims the benefit of the priority based on Korean Patent Application No. 10-2021-0065600 filed on May 21, 2021, and the entire contents disclosed in the document of the corresponding Korean patent application are incorporated as a part of the present description.

### TECHNICAL FIELD

The present application relates to a method for crystallization of an aromatic amino acid, enabling sustainable cycle of ammonia.

### BACKGROUND ART

L-tryptophan is one of essential amino acids, and most animals cannot form L-tryptophan by themselves, so it should be supplied by ingestion. Like L-tryptophan, as an aromatic amino acid, tyrosine, phenylalanine and histidine among 4 kinds of protein source amino acids correspond thereto. Amino acids including L-tryptophan have a role as a precursor for protein biosynthesis, and in case of L-tryptophan, it is converted into a substance essential for human health such as serotonin and niacin and the like through internal metabolism.

Aromatic amino acids have lower solubility in pure water than other amino acids. In other words, in case of aromatic amino acids including L-tryptophan in broth produced by an industrial fermentation method, they are present in a form of crystals at a fermentation concentration higher than the solubility. This may cause problems that reduce the recovery rate during the purification process, so it is needed to improve the process by increasing the solubility through introduction of an additive for crystal dissolution during the process.

A method for neutralization crystallization and a method for concentration crystallization used as a method for crystallizing an aromatic amino acid including L-tryptophan occur a risk of dropping quality due to a decrease in particle size and transition of impurities according to an increase of impurities. Therefore, a method which can minimize transition of impurifies during a crystallization process and improve a crystal particle size is needed.

A method for using a resin tower and a method for neutralization crystallization as a method for purifying L-tryptophan are less economical as a cost increase is caused due to the use of an additive during the process. On the other hand, when crystals are recovered through a concentration process after dissolving crystals by treatment with ammonia, production of high-content products is possible without addition of a separate process, and the ammonia recovered from the concentration process is economical as it is reused on a purpose of dissolving crystals.

Under these technical backgrounds, it has been confirmed that a process advantageous for quality and yield can be established through recrystallization after dissolving L-tryptophan in broth for purifying L-tryptophan, and based on this, the present application has been completed.

### PRIOR ART

### Patent Documents

(Patent document 1) KR 10-2016-0105827 A
(Patent document 2) CN 101914054 A
(Patent document 3) KR 10-2002-0086721 A
(Patent document 4) KR 10-1989-0001245 A

### DISCLOSURE

### Technical Problem

One object of the present application is to provide a method for crystallization of an aromatic amino acid, enabling sustainable cycle of ammonia.

Another object of the present application is to provide an aromatic amino acid crystal produced by the method.

### Technical Solution

Each description and embodiment disclosed in the application can be applied also to each other description and embodiment. In other words, all combinations of various elements disclosed in the present application fall within the scope of the present application. In addition, the scope of the present application cannot be considered limited by detailed description described below.

The present application provides a method for crystallization of an aromatic amino acid, comprising
(a) mixing a reaction solution comprising aromatic amino acid crystals and ammonia to obtain a dissolved solution in which the aromatic amino acid crystals are dissolved (crystal dissolution);
(b) crystallizing the obtained dissolved solution to obtain a concentrated solution comprising aromatic amino acid crystals (concentration crystallization);
(c) recovering gas comprising ammonia, produced during the process of crystallizing; and
(d) reusing ammonia derived from the recovered gas as ammonia of the step (a) as one aspect.

The method may further comprise the step (a) and the step (b), after the step (d).

The step (b) and step (c) may be conducted simultaneously or sequentially.

The step (a) to step (d) may be characterized by being performed repeatedly.

The method for crystallization of an aromatic amino acid of the present application is as follows, when described in detail at each step.

### Step (a)

First of all, the method of the present application may comprise the step (a), mixing (adding) a reaction solution comprising aromatic amino acid crystals and ammonia to obtain a dissolved solution in which the aromatic amino acid crystals are dissolved (crystal dissolution).

In the present application, the term, “aromatic amino acid” may be at least one selected from the group consisting of tryptophan, tyrosine, phenylalanine and histidine, and may be at least one selected from the group consisting of L-tryptophan, L-tyrosine, L-phenylalanine and L-histidine, and in one embodiment, it may be L-tryptophan.

In the above step, addition of ammonia can increase the pH of the reaction solution and/or culture solution comprising aromatic amino acid crystals, and increase the solubility of the aromatic amino acids in the reaction solution. In the ammonia, to produce a crystallization feed in the first process, separate ammonia, for example, ammonia water of 20 to 30% (v/v), 20 to 28% (v/v), 20 to 26% (v/v), 20 to 24% (v/v), 20 to 22% (v/v), 22 to 30% (v/v), 22 to 28% (v/v), 22 to 26% (v/v), 22 to 24% (v/v), 24 to 30% (v/v), 24 to 28% (v/V), 24 to 26% (v/v), 26 to 30% (v/v), 26 to 28% (v/v), or 28 to 30% (v/v), for example, ammonia water of 26% (v/v) may be added, and in the subsequent process, ammonia derived from the gas recovered from the step (c) may be added.

In one embodiment, the pH of the dissolved solution in which the aromatic amino acid crystals obtained in the step (a) are dissolved may be 10 to 12. When the pH of the dissolved solution is 10 or less, aromatic amino acid crystals are present in the dissolved solution, so the crystallization efficiency of the dissolved solution may be reduced. The pH of the aromatic amino acid dissolved solution may have a range of for example, 10 to 12, 10 to 11.5, 10 to 11, 10 to 10.5, 10.5 to 12, 10.5 to 11.5, 10.5 to 11, 11 to 12, 11 to 11.5, or 11.5 to 12, but it may be appropriately adjusted according to the kind of the amino acid, other reaction conditions, and the like. The concentration of ammonia added to the reaction solution and/or culture solution comprising the aromatic amino acid crystals may be used at a level to satisfy the pH of the reaction solution and/or culture solution within the above range.

In one embodiment, the dissolved solution in which the aromatic amino acid crystals are dissolved may be changed into a form of transparent solution in suspension as the aromatic amino acid crystals are dissolved.

In one embodiment, the reaction solution comprising aromatic amino acid crystals is a solution in which aromatic amino acids are supersaturated and may be in a form of suspension, and for example, it may include a solution or fermented solution comprising aromatic amino acid crystals.

The solution comprising aromatic amino acid crystals may be a solution in which aromatic amino acid crystals and distilled water are mixed, and the fermented solution comprising aromatic amino acid crystals may be obtained by culturing a microorganism producing an aromatic amino acid in broth.

The microorganism producing an aromatic amino acid is not particularly limited as long as it is a microorganism with production ability of an aromatic amino acid, and for example, it may be the genus of Corynebacterium or the genus of Escherichia, and specifically, it may be a strain of Corynebacterium glutamicum or a mutant thereof.

In the present application, the term, “culture” means growing a microorganism under an environmental condition adjusted moderately artificially. In the present application, the method for producing an aromatic amino acid using a microorganism having production ability of an aromatic amino acid may be performed using a method widely known in the art. Specifically, the culture may be continuously culturing in a batch process, fed batch or repeated fed batch process, but not limited thereto.

Broth used for culture should satisfy requirements of a specific strain in an appropriate method. For example, culture broth for a Corynebacterium sp. strain is known (for example, Manual of Methods for General Bacteriology. American Society for Bacteriology. Washington D.C., USA, 1981). As a usable sugar source, a sugar and a carbohydrate such as glucose, saccharose, lactose, fructose, maltose, starch and cellulose, oil such as soybean oil, sunflower oil, castor oil, coconut oil and the like, and a fatty acid such as fat, palmitic acid, stearic acid, and linoleic acid, and an alcohol such as glycerol and ethanol, and an organic acid such as acetic acid may be comprised. These substances may be used separately or in a mixture, but not limited thereto. As a usable nitrogen source, peptone, yeast extract, meat juice, malt extract, corn steep liquid, soybean meal and urea or an inorganic compound, for example, ammonium sulfate, ammonium chloride, ammonium phosphate, ammonium carbonate and ammonium nitrate may be comprised. The nitrogen source may be also used separately or in a mixture, but not limited thereto. As a usable phosphorus source, potassium dihydrogen phosphate or dipotassium hydrogen phosphate or a corresponding sodium-containing salt may be comprised.

In addition, the culture broth may contain a metal salt such as magnesium sulfate or iron sulfate required for growth. Additionally, an essential growth substance such as an amino acid and a vitamin may be used. Moreover, precursors suitable for culture broth may be used. The raw materials described above may be added in a batch manner or a continuous manner by a method suitable for cultures during a culture process. However, it is not limited thereto.

### Step (b)

After that, the method of the present application may comprise the step (b), crystallizing a dissolved solution in which the obtained aromatic amino acid crystals are dissolved to obtain a concentrated solution comprising aromatic amino acid crystals (concentration crystallization).

In the step, a technology known in the art may be applied to crystallization of an aromatic amino acid dissolved solution. For example, the crystallization may accompany evaporation of a solvent and vapor, and during progressing the crystallization process, the temperature may be 60 to 90° C., 60 to 85° C., 60 to 80° C., 60 to 75° ° C., 60 to 70° C., 60 to 65° C., 65 to 90° C., 65 to 85° C., 65 to 80° C., 65 to 75° C., 65 to 70° ° C., 70 to 90° C., 70 to 85° C., 70 to 80° C., 70 to 75° ° C., 75 to 90° C., 75 to 85° C., 75 to 80° C., 80 to 90° C., 80 to 85° ° C., or 85 to 90° C., for example, 80° C., but not limited thereto. Furthermore, during performing the crystallizing, a stirring process may be further performed, and the stirring speed may be 50 to 500 rpm, 50 to 400 rpm, 50 to 300 rpm, 50 to 200 rpm, 50 to 150 rpm, 50 to 100 rpm, 100 to 500 rpm, 100 to 400 rpm, 100 to 300 rpm, 100 to 200 rpm, 100 to 150 rpm, 150 to 500 rpm, 150 to 400 rpm, 150 to 300 rpm, 150 to 200 rpm, 200 to 500 rpm, 200 to 400 rpm, or 200 to 300 rpm, for example, 200 rpm, but not limited thereto. In addition, during performing the crystallizing, a process of reducing internal pressure may be further performed, and the internal pressure during performing the crystallizing may be 10 to 500 mbar, 10 to 400 mbar, 10 to 300 mbar, 10 to 200 mbar, 10 to 100 mbar, 10 to 50 mbar, 50 to 500 mbar, 50 to 400 mbar, 50 to 300 mbar, 50 to 200 mbar, 50 to 100 mbar, 100 to 500 mbar, 100 to 400 mbar, 100 to 300 mbar, or 100 to 200 mbar, for example, 100 mbar, but not limited thereto.

The concentrated solution contains aromatic amino acids at a high concentration, and the concentration of the aromatic amino acids may be for example, 20 to 500 g/L, 20 to 400 g/L, 20 to 300 g/L, 20 to 250 g/L, 20 to 200 g/L, 20 to 180 g/L, 20 to 150 g/L, 20 to 100 g/L, 20 to 90 g/L, 20 to 80 g/L, 20 to 70 g/L, 20 to 60 g/L, 20 to 50 g/L, 20 to 40 g/L, 20 to 30 g/L, 30 to 500 g/L, 30 to 400 g/L, 30 to 300 g/L, 30 to 250 g/L, 30 to 200 g/L, 30 to 180 g/L, 30 to 150 g/L, 30 to 100 g/L, 30 to 90 g/L, 30 to 80 g/L, 30 to 70 g/L, 30 to 60 g/L, 30 to 50 g/L, 30 to 40 g/L, 40 to 500 g/L, 40 to 400 g/L, 40 to 300 g/L, 40 to 250 g/L, 40 to 200 g/L, 40 to 180 g/L, 40 to 150 g/L, 40 to 100 g/L, 40 to 90 g/L, 40 to 80 g/L, 40 to 70 g/L, 40 to 60 g/L, 40 to 50 g/L, 50 to 500 g/L, 50 to 400 g/L, 50 to 300 g/L, 50 to 250 g/L, 50 to 200 g/L, 50 to 180 g/L, 50 to 150 g/L, 50 to 100 g/L, 50 to 90 g/L, 50 to 80 g/L, 50 to 70 g/L, 50 to 60 g/L, 60 to 500 g/L, 60 to 400 g/L, 60 to 300 g/L, 60 to 250 g/L, 60 to 200 g/L, 60 to 180 g/L, 60 to 150 g/L, 60 to 100 g/L, 60 to 90 g/L, 60 to 80 g/L, 60 to 70 g/L, 70 to 500 g/L, 70 to 400 g/L, 70 to 300 g/L, 70 to 250 g/L, 70 to 200 g/L, 70 to 180 g/L, 70 to 150 g/L, 70 to 100 g/L, 70 to 90 g/L, 70 to 80 g/L, 80 to 500 g/L, 80 to 400 g/L, 80 to 300 g/L, 80 to 250 g/L, 80 to 200 g/L, 80 to 180 g/L, 80 to 150 g/L, 80 to 100 g/L, 80 to 90 g/L, 90 to 500 g/L, 90 to 400 g/L, 90 to 300 g/L, 90 to 250 g/L, 90 to 200 g/L, 90 to 180 g/L, 90 to 150 g/L, 90 to 100 g/L, 100 to 500 g/L, 100 to 400 g/L, 100 to 300 g/L, 100 to 250 g/L, 100 to 200 g/L, 100 to 180 g/L, 100 to 150 g/L, 150 to 500 g/L, 150 to 400 g/L, 150 to 300 g/L, 150 to 250 g/L, 150 to 200 g/L, 150 to 180 g/L, 180 to 500 g/L, 180 to 400 g/L, 180 to 300 g/L, 180 to 250 g/L, 180 to 200 g/L, 200 to 500 g/L, 200 to 400 g/L, 200 to 300 g/L, 200 to 250 g/L, 250 to 500 g/L, 250 to 400 g/L, 250 to 300 g/L, 300 to 500 g/L, 300 to 400 g/L, or 400 to 500 g/L, but this may be changed appropriately depending on the scale of industrialization process.

In one embodiment, the concentration of the aromatic amino acids of the concentrated solution may be 1 to 3 times, 1 to 2.8 times, 1 to 2.5 times, 1 to 2.2 times, 1 to 2 times, 1 to 1.9 times, 1 to 1.8 times, 1 to 1.7 times, 1 to 1.6 times, 1 to 1.5 times, 1 to 1.4 times, 1 to 1.3 times, 1 to 1.2 times, 1 to 1.1 times, 1.1 to 3 times, 1.1 to 2.8 times, 1.1 to 2.5 times, 1.1 to 2.2 times, 1.1 to 2 times, 1.1 to 1.9 times, 1.1 to 1.8 times, 1.1 to 1.7 times, 1.1 to 1.6 times, 1.1 to 1.5 times, 1.1 to 1.4 times, 1.1 to 1.3 times, 1.1 to 1.2 times, 1.2 to 3 times, 1.2 to 2.8 times, 1.2 to 2.5 times, 1.2 to 2.2 times, 1.2 to 2 times, 1.2 to 1.9 times, 1.2 to 1.8 times, 1.2 to 1.7 times, 1.2 to 1.6 times, 1.2 to 1.5 times, 1.2 to 1.4 times, 1.2 to 1.3 times, 1.3 to 3 times, 1.3 to 2.8 times, 1.3 to 2.5 times, 1.3 to 2.2 times, 1.3 to 2 times, 1.3 to 1.9 times, 1.3 to 1.8 times, 1.3 to 1.7 times, 1.3 to 1.6 times, 1.3 to 1.5 times, 1.3 to 1.4 times, 1.4 to 3 times, 1.4 to 2.8 times, 1.4 to 2.5 times, 1.4 to 2.2 times, 1.4 to 2 times, 1.4 to 1.9 times, 1.4 to 1.8 times, 1.4 to 1.7 times, 1.4 to 1.6 times, 1.4 to 1.5 times, 1.5 to 3 times, 1.5 to 2.8 times, 1.5 to 2.5 times, 1.5 to 2.2 times, 1.5 to 2 times, 1.5 to 1.9 times, 1.5 to 1.8 times, 1.5 to 1.7 times, 1.5 to 1.6 times, 1.6 to 3 times, 1.6 to 2.8 times, 1.6 to 2.5 times, 1.6 to 2.2 times, 1.6 to 2 times, 1.6 to 1.9 times, 1.6 to 1.8 times, 1.6 to 1.7 times, 1.7 to 3 times, 1.7 to 2.8 times, 1.7 to 2.5 times, 1.7 to 2.2 times, 1.7 to 2 times, 1.7 to 1.9 times, 1.7 to 1.8 times, 1.8 to 3 times, 1.8 to 2.8 times, 1.8 to 2.5 times, 1.8 to 2.2 times, 1.8 to 2 times, 1.8 to 1.9 times, 1.9 to 3 times, 1.9 to 2.8 times, 1.9 to 2.5 times, 1.9 to 2.2 times, 1.9 to 2 times, 2 to 3 times, 2 to 2.8 times, 2 to 2.5 times, 2 to 2.2 times, 2.2 to 3 times, 2.2 to 2.8 times, 2.2 to 2.5 times, 2.5 to 3 times, 2.5 to 2.8 times, or 2.8 to 3 times based on the concentration of the aromatic amino acids of the reaction solution comprising aromatic amino acid crystals of the step (a), but not limited thereto.

In one embodiment, the concentrated solution may be changed into a form of suspension as the turbidity of solution is increased as aromatic amino acid crystals are formed again by crystallization of a transparent aromatic amino acid dissolved solution.

### Step (c)

After that, the method of the present application may comprise the step (c), recovering (obtaining) gas comprising ammonia, produced during the process of crystallizing.

The gas comprising ammonia of the step (c) may be mixed gas comprising ammonia and vapor.

In one embodiment, the step (c) may be conducted simultaneously or sequentially with the aforementioned step (b) which obtains a concentrated solution comprising aromatic amino acid crystals.

In one embodiment, the step may be conducted until the pH of the dissolved solution in which aromatic amino acid crystals of the aforementioned step (b) are dissolved becomes 5.5 to 8, and specifically, it may be conducted until the pH of the dissolved solution becomes 5.5 to 8, 5.5 to 7.5, 5.5 to 7, 5.5 to 6.5, 5.5 to 6, 6 to 8, 6 to 7.5, 6 to 7, 6 to 6.5, 6.5 to 8, 6.5 to 7.5, 6.5 to 7, 7 to 8, 7 to 7.5, or 7.5 to 8, but not limited thereto. When the pH of the dissolved solution is within the above range, for example, less than 8, the concentration of ammonia in the obtained mixed gas is low, so the crystallization efficiency of the aromatic amino acid can be reduced by decreasing the concentration of the recovered ammonia.

In addition, the gas comprising ammonia may be gas in a vapor state in which vapor and ammonia are mixed, and the composition ratio of the mixed gas is predominantly influenced by the gas-liquid phase equilibrium system of water-ammonia-aromatic amino acid, so it may have various distributions depending on the internal temperature and pressure conditions of a crystallization device.

### Step (d)

Then, the method of the present application may comprise reusing ammonia derived from the recovered (obtained) gas as ammonia of the step (a) to obtain a dissolved solution in which aromatic amino acid crystals are dissolved again.

In one embodiment, the step may use the ammonia in a vapor state, or reuse it in a state in which the vapor is compressed or condensed into a liquid state.

Before the step, it may further comprise converting ammonia in gas into a liquid state, by compressing or condensing the recovered gas, and for example, it may condense mixed gas using a condenser, or compress mixed gas using a compressor, or use the condenser and compressor at the same time.

In one embodiment, the step may be performed repeatedly until a concentrated solution comprising a large amount of aromatic amino acid crystals at a high concentration can be obtained. For example, the step may be repeatedly performed once or more, twice or more, 3 times or more, 4 times or more, 5 times or more, or 2 to 50 times, 2 to 45 times, 2 to 40 times, 2 to 35 times, 2 to 30 times, 2 to 25 times, 2 to 20 times, 2 to 15 times, 2 to 10 times, or 2 to 5 times, but not limited thereto.

The method of the present application is economical as it does not require a large amount of pH adjusting substances, and can improve the aromatic amino acid crystallization efficiency, by comprising reusing ammonia derived from the recovered gas in the step (c) as ammonia of the step (a), and can be used as an eco-friendly method by reducing production of salt waste of which sustainable cycle is difficult.

In addition, the method of the present application may comprise separating the crystals from the dissolved solution in which the aromatic amino acid crystals are dissolved using centrifugation equipment, after the step (b).

According to one example, ammonia produced during multiple crystallization processes can be obtained at a high level of recovery rate, and the recovered ammonia, for example, ammonia water is reused for dissolving crystals of aromatic amino acids present in the reaction solution, thereby increasing economic feasibility. Therefore, the method can continuously produce crystals of aromatic amino acids, based on the crystallization process without a separate pH adjusting agent, and thus, aromatic amino acid production efficiency can be improved.

The method of the present application may further comprise separating aromatic amino acid crystals from the concentrated solution comprising aromatic amino acid crystals of the step (b). The separating may comprise centrifugation and/or filtration processes. The separating aromatic amino acid crystals, may be conducted between the step (b) and step (c), between the step (c) and step (d), or after the step (d), and may be conducted at the same time with the step (C) or step (d), or may be conducted at any stage regardless of the order of the step (c) and step (d) as long as it is after the step (b).

In the step, an example of separation of aromatic amino acid crystals from the concentrated solution, may comprise for example, a solid-liquid separating concentrated solution comprising the aromatic amino acid crystals to obtain aromatic amino acid wet crystals; and drying the obtained wet crystals to obtain aromatic amino acid crystals, but a conventional technology known in the art, related to separation and purification of amino acid crystals, may be applied non-restrictively.

After that, the method of the present application may comprise crystallizing the dissolved solution obtained in the step (d) to obtain a concentrated solution comprising aromatic amino acid crystals (concentration recrystallization).

In one embodiment, the step (a) to step (d) may be performed repeatedly once or more, twice or more, 3 times or more, 4 times or more, 5 times or more, or 2 to 50 times, 2 to 45 times, 2 to 40 times, 2 to 35 times, 2 to 30 times, 2 to 25 times, 2 to 20 times, 2 to 15 times, 2 to 10 times, or 2 to 5 times, but not limited thereto.

In addition, the present application provides an aromatic amino acid crystal, produced by a method for crystallization of an aromatic amino acid comprising (a) mixing a reaction solution comprising aromatic amino acid crystals and ammonia to obtain a dissolved solution in which the aromatic amino acid crystals are dissolved (crystal dissolution);
(b) crystallizing the obtained dissolved solution to obtain concentrated solution comprising aromatic amino acid crystals (concentration crystallization);
(c) recovering gas comprising ammonia, produced during the process of crystallizing; and
(d) reusing ammonia derived from the recovered gas as ammonia of the step (a) as one aspect.

The aromatic amino acid and method for crystallization of an aromatic amino acid are as described above.

### Advantageous Effects

The method for crystallization of an aromatic amino acid according to the present application can improve production efficiency of aromatic amino acid crystals, by recovering ammonia initially added for increasing solubility of an aromatic amino acid in a gas state and reusing it, and can reduce production costs as a large amount of pH adjusting substances and an additional neutralization process are not required.

In addition, aromatic amino acid crystals with excellent particle size can be formed by recrystallization through progressing concentration crystallization after dissolving aromatic amino acid crystals by treatment of ammonia. The aromatic amino acid crystals formed thereby can be recovered in a wet tablet form by separation.

### BRIEF DESCRIPTION OF THE DRAWINGS

FIG. 1 is a drawing which schematically shows the process of crystallization of L-tryptophan and recovery of ammonia according to one example.

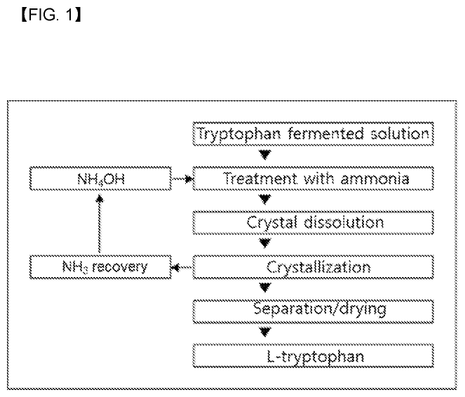

FIG. 2 is the result of confirming the change in solubility of L-tryptophan depending on pH of solution.

FIG. 3 is a drawing which schematically shows the recrystallization device and ammonia recovery device according to one example.

### MODE FOR INVENTION

Hereinafter, the present invention will be described in more detail by examples. However, these examples are intended to illustratively describe the present invention, but the scope of the present invention is not limited by these examples. In addition, contents not described in the present description can be fully recognized and inferred by those skilled in the technical field of the present application or a similar field, so description thereof is omitted.

### Example 1. Experimental Materials and Experimental Method

### (1) Experimental Materials

As an aromatic amino acid, L-tryptophan, products with a purity of 98% or more were used, and all CJ Cheil Jedang products were used. As water, tertiary distilled water directly prepared was used, and 26% (v/v) ammonia water was purchased from Daejungchem company and used. A 0.1 M nitrate aqueous solution and a 0.02M dipicolinic acid aqueous solution for high-performance liquid chromatograph (HPLC) were purchased from Sigma-Aldrich (US) and used.

### (2) Analysis of Concentration of L-Tryptophan Dissolved Solution and Purity of L-Tryptophan Crystals

Analysis of the concentration of the L-tryptophan dissolved solution and purity of L-tryptophan crystals was performed using high-performance liquid chromatography (chromatography C18), and the analysis conditions are as follows:
Column: Hypersil gold HPLC column (Thermo Scientific, US)
Column temperature: 35° C.
Mobile phase: Potassium phosphate 7.35 mM & Acetonitrile solution
Mobile phase velocity: 1.5 ml/min
Detector: UV detector.

### (3) Analysis of Concentration of Ammonia in L-Tryptophan Dissolved Solution

Analysis of the concentration of ammonia in the L-tryptophan dissolved solution was performed using ion chromatography (model 930 compact IC Flex, Metrohm, Switzerland).
Column: Metrosep C4-150 (Metrohm, Switzerland)
Column temperature: 25° C.
Mobile phase: 0.7 mM nitrate aqueous solution+1.7 mM dipicolinic acid aqueous solution
Mobile phase velocity: 1.0 ml/min.

### (4) Analysis of Solubility of L-Tryptophan

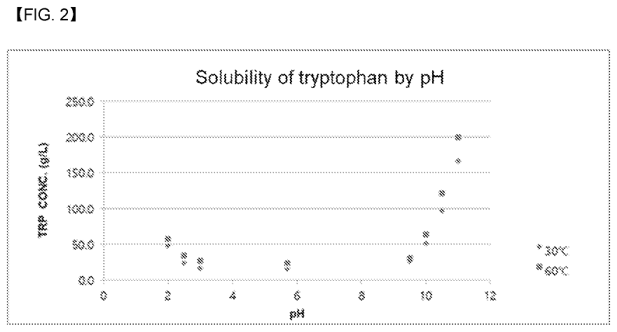

Measurement of solubility of L-tryptophan amino acids was performed in a 1 L jacket reactor made of glass. After mixing distilled water and ammonia water in the 1 L jacket reactor at various ratios, an excess of L-tryptophan crystals was added and they were stirred, thereby preparing a reaction solution comprising L-tryptophan crystals. After that, while maintaining the internal temperature of the reactor at 30° C. constantly using freezing/heating circulation apparatus (model F35, Julabo, Germany), these conditions were applied with stirring for 12 hours or more. Then, when the stirring was stopped and all L-tryptophan crystals were sunk, a part of clear supernatant was transferred to a syringe sampler equipped with a 0.45 μm syringe filter. At this time, the pH of the corresponding solution was measured suing a pH measuring instrument (model S220, Mettler Toledo, US). The concentration of the corresponding sample was measured using HPLC, after diluting with tertiary distilled water using a volumetric flask. The concentration of the supernatant sample was assumed with the solubility, and the change in solubility of L-tryptophan depending on pH of solution was shown in FIG. 2. In other words, the pH of the solution could be confirmed that the solubility of L-tryptophan increased from the time when the pH of the solution was to be 10 or more.

### Example 2. Separation and Purification from Solution Comprising L-Tryptophan Crystals

### (1) Crystallization of L-Tryptophan

Crystallization of L-tryptophan was performed from the reaction solution treated with ammonia in a culture solution. After conducting crystal dissolution through treatment of ammonia to the culture solution containing L-tryptophan crystals, recrystallization was performed by concentration crystallization, thereby passing through a step of forming crystals advantageous for the particle size and quality. Ammonia treated to the culture solution was ammonia water of 26% (v/v), and by treating ammonia, the pH of reaction solution was adjusted to a range of 10 to 12.

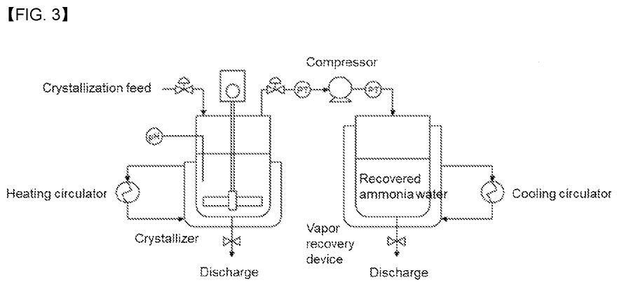

The schematic diagram of the crystallization device and ammonia recovery device according to one example is as shown in FIG. 3. Specifically, in FIG. 3, the crystallization feed means a concentrated solution comprising L-tryptophan crystals, obtained in the first-round crystallization process, and crystallization was progressed in a 20 L jacket reactor made of glass, and in order to prevent scaling in a heat exchanger, the jacket was installed only up to the height where the volume of the internal liquid was 10 L. The jacket region was filled with tertiary distilled water in which temperature was controlled with freezing/heating circulation apparatus (model F35, Julabo, Germany), and the temperature was maintained at 80° C. during performing the L-tryptophan crystallization process. Stirring of the internal reaction solution and the like was performed using a stirrer capable of adjusting the rotation velocity with a 4-blade impeller made of Teflon (model RW-20, IKA, Germany), and the stirring velocity during progressing crystallization was maintained at 200 rpm. A decrease in pressure inside the reactor was carried out using a compressor connected with a pipe, and an electronic vacuum controller for adjustment of pressure (model NVC 2300-A, Eyela, Japan) was installed between the compressor and crystallizer, and pressure thereof was adjusted. The internal pressure of the crystallizer was maintained at 100 mbar. The ammonia recovery unit consisted of a 20 L jacket pressure container made of stainless steel capable of storing up to 20 bars, and cooling was performed with cooling water of 4° C. supplied from its own equipment. At this time, the recovery of ammonia was performed until the pH inside the crystallizer became less than 8, and at pH lower than this, the concentration of the recovered ammonia was low, so recovery of ammonia was not carried out. The concentration of the final concentrated solution was adjusted so that it became 3 times of the concentration of the reaction solution comprising L-tryptophan crystals before adding ammonia.

### (2) Separation and Drying of L-Tryptophan Crystals

When the final concentrated concentration was reached, the crystallization process was terminated and the concentrated solution in the reactor was recovered through a discharge pipe placed on the bottom. After that, it was under solid-liquid separation at a speed of 4000 rpm using a high centrifugal decanter. If necessary, a washing process was performed at the beginning of separation using distilled water. Then, the obtained wet crystals were dried in an oven drier of 80° C. until there was no change in weight to obtain crystals of L-tryptophan.

### (3) Experimental Result: Recrystallization of L-Tryptophan and Recovery of Ammonia

Experiments of crystallization of L-tryptophan and ammonia recovery were performed using 20 L suspension prepared with L-tryptophan crystals with a purity of 98% or more.

Crystallization was carried out 5 times in total under the same process conditions for each condition. However, in the first-round crystallization process, to prepare the crystallization feed, by adding 26% (v/v) ammonia water to the solution comprising L-tryptophan crystals prepared by mixing L-tryptophan crystals and distilled water, the pH of the reaction solution was adjusted to a range of 10 to 12, and the entire amount of L-tryptophan crystals was dissolved. In the subsequent round, by adding recovered ammonia water to the reaction solution comprising L-tryptophan crystals, the L-tryptophan crystals were dissolved (experimental group). On the other hand, as a control group, a group in which ammonia water of 26% (v/v) was added to the reaction solution comprising L-tryptophan crystals without an ammonia water recovery process, and then the pH was readjusted to 7 with sulfuric acid of 98% (v/v) was used.

The experiment was performed 5 times in total, and the average value for the crystallization processes from the second round to the fifth round of the corresponding experimental results was shown in Table 1 below. For the reaction solution with a concentration range of L-tryptophan of 15 to 80 g/L, the recovered ammonia was added, and the pH of the reaction solution was adjusted to a range of 9 to 12, thereby dissolution of crystals was performed, and then while conducting crystallization, ammonia water was recovered again.

As shown in Table 1 below, there was no recovered ammonia water in the control group, but in the crystallization process according to one example, ammonia water at a concentration of 5 to 23% could be secured at a recovery rate at a level of 90%, and the recovered ammonia water could be reused for dissolving crystals of L-tryptophan in the reaction solution.

TABLE 1

| Item | Control group | | | Experimental group | | |
|---|---|---|---|---|---|---|
| Initial reaction solution | 42 | 45 | 52 | 68 | 48 | 77 |
| concentration (g/L) | | | | | | |
| Initial reaction solution pH | 6.2 | 6.5 | 6.0 | 6.0 | 6.4 | 6.2 |
| Recovered ammonia feed | 0 | 0 | 0 | 10 | 10 | 10 |
| ratio (vol %) | | | | | | |
| 26% ammonia feed ratio | 8 | 9 | 10 | 0 | 0 | 0 |
| (vol %) | | | | | | |
| 98% sulfate feed ratio | 4 | 5 | 5 | 1 | 2 | 2 |
| (vol %) | | | | | | |
| Concentration of obtained | 42 | 45 | 52 | 62 | 44 | 71 |
| concentrated solution during | | | | | | |
| first crystallization process | | | | | | |
| (g/L) | | | | | | |
| pH of concentrated solution | 6.2 | 6.5 | 6.0 | 9.4 | 10 | 10.5 |
| during first round | | | | | | |
| crystallization process | | | | | | |
| Final concentrated solution | 180 | 180 | 180 | 180 | 180 | 180 |
| concentration (g/L) | | | | | | |
| Final concentrated solution | 6.2 | 6.5 | 6.0 | 7.5 | 8.0 | 7.8 |
| pH | | | | | | |
| Crystal recovery rate (%) | 72 | 76 | 78 | 86 | 90 | 95 |
| Ammonia recovery rate (%) | 0 | 0 | 0 | 90 | 90 | 92 |
| Recovered ammonia | 0 | 0 | 0 | 14 | 15 | 17 |
| concentration (%) | | | | | | |

### Example 3. Separation and Purification in Fermented Solution Comprising L-Tryptophan Crystals

### (1) Preparation of Fermented Solution Comprising L-Tryptophan

Using a strain producing L-tryptophan, Corynebacterium KCCM12218P (Korean Patent Application No. 10-2018-0022057), a fermented solution comprising L-tryptophan was prepared.

Specifically, preculture broth 40 mL was aliquoted in a 500 mL Erlenmeyer flask for shaking, and was under pressure sterilization at 121° C. for 15 minutes, and then the strain was phagocytized and cultured in a rotary shaking incubator for 24 hours while stirring at 200 rpm at 33° C. Then, seed-culture broth 3 L was filled in a 5 L fermenter, and was under pressure sterilization at 121° ° C. for 30 minutes, and then pH was adjusted to 7.0, and the preculture 4% was phagocytized and cultured until the OD value was 20 under conditions of 800 rpm and quantity of airflow of 0.5 vvm at 33° C. to conduct seed-culture. After that, main culture broth 2.1 L was filled in a 5 L fermenter and was under pressure sterilization at 121° C. for 30 minutes, and then glucose was added by 0.6 L, and using ammonia gas, pH was adjusted to 7.0. The seed-culture was phagocytized in a main culture container to 20%, and was cultured for 42 hours while adjusting it so that dissolved oxygen was maintained at least 30% under conditions of the culture temperature of 33° C. and quantity of airflow of 1.0 vvm, thereby preparing each fermented solution comprising L-tryptophan. The compositions of the preculture, seed culture and main culture broth used in the culture process were as Table 2 below.

TABLE 2

| | Preculture | Seed culture | Main culture |
|---|---|---|---|
| Composition | broth | broth | broth |
| Glucose (g/l) | 20 | 20 | 20 |
| Peptone (g/l) | 10 | 10 | 10 |
| Yeast extracted solution (g/l) | 5 | 5 | 5 |
| Urea (g/l) | 1.5 | 1.5 | 1.5 |
| KH2PO4 (g/l) | 4 | 4 | 4 |
| K2HPO4 (g/l) | 8 | 8 | 8 |
| MgSO4 7H2O(g/l) | 0.5 | 0.5 | 0.5 |
| Biotin (ug/l) | 100 | 100 | 100 |
| Thiamine HCl (ug/l) | 1000 | 1000 | 1000 |
| Calcium-pantothenic acid (ug/l) | 2000 | 2000 | 2000 |
| Nicotinamide (ug/l) | 2000 | 2000 | 2000 |

### (2) Separation and Drying of L-Tryptophan Crystals

Using the fermented solution comprising L-tryptophan crystals prepared above instead of the solution comprising L-tryptophan crystals, by the same method as described in (1) and (2) of Example 2, addition of ammonia water (26% (v/v)), crystallization, and separation and drying of L-tryptophan crystals were performed. Decanter equipment using a centrifugal force for the method for separation of crystals was used, and the centrifugal force was progressed at a level of 4000 rpm. If necessary, washing was performed by adding water.

### (3) Experimental Result: Purification of L-Tryptophan

From the fermented solution at a concentration of L-tryptophan of 60 g/L comprising L-tryptophan crystals, prepared in (1) of Example 3, purification of L-tryptophan was conducted 5 times repeatedly. Recovered ammonia water (however, ammonia water reagent at first) was added to the fermented solution 20 L comprising L-tryptophan to adjust pH to 10, and a whole quantity of L-tryptophan crystals was dissolved, and then concentration crystallization was performed to a concentration of 180 g/L, and the concentrated solution comprising L-tryptophan crystals was under solid-liquid separation using centrifugation equipment. Then, washing was conducted using tertiary distilled water of 20 vol % if needed.

Then, the weight of the crystals finally recovered by drying was 1.1 kg on average of 5 times, and the purity of crystals was 98.2% on average of 5 times. In the crystallization process according to one example, ammonia water of 13% concentration could be secured at a recovery rate of 90% or more, and the recovered ammonia water could be reused for dissolving the crystals of L-tryptophan in the fermented solution.

The description of the present invention described above is for illustration, and those skilled in the art to which the present invention pertains can understand that it can be easily modified into other specific forms without changing the technical spirit or essential features of the present invention. Therefore, the examples described above should be understood as illustrative and not restrictive in all aspects.

1. A method for crystallization of an aromatic amino acid, comprising
(a) mixing a reaction solution comprising aromatic amino acid crystals and ammonia to obtain a dissolved solution in which the aromatic amino acid crystals are dissolved;
(b) crystallizing the obtained dissolved solution to obtain a concentrated solution comprising aromatic amino acid crystals;
(c) recovering gas comprising ammonia, produced during the process of crystallizing; and
(d) reusing ammonia derived from the recovered gas as ammonia of the step (a);
wherein the step (b) and step (c) are conducted simultaneously or sequentially, and
the step (a) to step (d) are performed repeatedly.

2. The method for crystallization of an aromatic amino acid according to claim 1, wherein the aromatic amino acid is at least one amino acid selected from the group consisting of L-tryptophan, L-tyrosine, L-phenylalanine, and L-histidine.

3. The method for crystallization of an aromatic amino acid according to claim 1, wherein the reaction solution comprising aromatic amino acid crystals comprises a solution or fermented solution comprising aromatic amino acid crystals.

4. The method for crystallization of an aromatic amino acid according to claim 3, wherein the fermented solution comprising aromatic amino acid crystals is obtained by culturing a microorganism producing an aromatic amino acid in broth.

5. The method for crystallization of an aromatic amino acid according to claim 1, wherein the pH of the dissolved solution in which the aromatic amino acid crystal is dissolved is 10 to 12, in the step (a).

6. The method for crystallization of an aromatic amino acid according to claim 1, wherein the concentration of the aromatic amino acid of the concentrated solution comprising crystal of the aromatic amino acid of the step (b) is 1.3 to 3 times based on the concentration of the aromatic amino acid of the reaction solution comprising crystal of the aromatic amino acid of the step (a).

7. The method for crystallization of an aromatic amino acid according to claim 1, wherein the step (c) is performed until the pH of the dissolved solution of the aromatic amino acid of the step (b) is less than 8.

8. The method for crystallization of an aromatic amino acid according to claim 1, wherein the step (d) reuses the ammonia in a vapor state, or reuses the vapor in a compressed or condensed liquid state.

9. An aromatic amino acid crystal, produced by the method for crystallization of an aromatic amino acid of claim 1.

10. The aromatic amino acid crystal according to claim 9, wherein the aromatic amino acid is at least one amino acid selected from the group consisting of L-tryptophan, L-tyrosine, L-phenylalanine, and L-histidine.
